(19) United States
(12) Patent Application Publication
(10) Pub. No.: US 2024/0404711 A1
(43) Pub. Date: Dec. 5, 2024

(54) IoT Based Dynamic Risk Assessment in Intelligent Building

(71) Applicant: Amrita Vishwa Vidyapeetham, Kerala (IN)

(72) Inventors: Divya Saraswathy Jayaprakash, Kerala (IN); Aryadevi Remanidevi Devidas, Kerala (IN); Maneesha Vinodini Ramesh, Kerala (IN)

(73) Assignee: Amrita Vishwa Vidyapeetham, Kerala (IN)

(21) Appl. No.: 18/358,346

(22) Filed: Jul. 25, 2023

(30) Foreign Application Priority Data
Jun. 2, 2023 (IN) 202341037904

(51) Int. Cl. G16H 50/80

(52) U.S. Cl. CPC G16H 50/80; G16Y 10/60

(57) ABSTRACT

A cloud-based, three-layered IoT-enabled, dynamic assessment and prediction system RAIB (Risk Assessment in Intelligent Building) in an intelligent building (IB) is disclosed, comprising of Risk assessment module I (RAIB-I), capable of assessing the risk based on the primary risk factors (fp1, fp2, fp3, . . . fpn) obtained from multiple users (q1, q2, q3, . . . qn) in the said building, and Risk assessment module II (RAIB-II), capable of assessing the risk factors based on the secondary risk factors (fs1, fs2 . . . fsn) of the said multiple users (q1, q2, q3, . . . qn). Prediction of future risk is done by prediction-based model using the historical risk assessment data. The Cloud layer (CL) deployed with RAIB and risk prediction module is used for performing the two-phase risk assessment, RAIB-I and RAIB-II and risk prediction from the stored historical data (HD).

### CROSS-REFERENCE TO RELATED APPLICATIONS

The instant application claims priority to Indian application Ser. No. 20/234,1037904, filed Jun. 2, 2023. All disclosure of the parent application is incorporated at least by reference.

### BACKGROUND OF THE INVENTION

1. Field of the Invention

The present invention is in the technical field of dynamic, real-time, cloud-based, IoT-enabled dynamic risk assessment and prediction of contagious diseases in an intelligent building. More particularly, the present invention relates to a two-phase, three-layered dynamic risk assessment and prediction system to assess as well as predict the risk of spread of contagious disease in an intelligent building, on the basis of primary and secondary risk factors.

2. Description of Related Art

The world is facing new airborne contagious diseases and their mutants day by day. Such diseases result in significant loss of human life worldwide, economic and social disruption, and public health challenges. As per a recent report of the World Health Organization (WHO), contagious diseases kill over 17 million people a year. This data becomes all the more significant, in view of the recent COVID-19 pandemic which devastated the world due to the strong contagious characteristics of the virus. Therefore, in order to ensure the safety of humankind, assessing the risk of airborne contagious diseases is vital.

Since the cities are highly populated, the chances of contagious disease spread in cities is higher when compared to rural regions. Moreover, enclosed spaces are more common in cities as compared to rural areas, due to industrialization, lifestyle differences and work habits, which facilitates the spread of contagious infections. In a building, the risk of spread of contagious diseases is higher than in an open area because there is more possibility of spreading the disease in a closed space.

Intelligent buildings (IBs) are enclosed entities in a smart city where communication exists between the individuals, the sensor systems, and the Internet of Things (IoT) devices of the building. The Intelligent building is designed to integrate IoT-connected devices, cloud computing, augmented reality, and other systems into a platform that automates day-to-day processes. These are the facilities that leverage complex automated systems to maximize operational efficiency and the well-being of occupants.

Intelligent buildings (IBs) have many floors and different sections for proper functioning. Apart from these sections, several common areas such as the reception, waiting area, corridor, parking area, cafeteria etc. can be found in an intelligent building, with a chance of entry for anyone in the building. Therefore, it is important to evaluate the risk of contagious diseases spread in intelligent buildings. However, the risk assessment of contagious diseases spread in the common areas is complex since everyone has access to these areas, and restrictions of entry in these areas and social distancing are nearly impractical.

Studies have been ongoing to develop systems for assessing the risk of contagious diseases spread in enclosed buildings of cities, by using the data acquired by the interconnected devices, individuals, and systems in the said buildings and other such enclosed spaces.

Many researchers have undertaken studies on infectious diseases including airborne contagious disease. However, risk assessment of such infectious disease is rarely proposed. Such studies have become important in view of the fact that in locations where everyone has access, and entry restriction is almost impractical, there is a need to assess the risk of spread of contagious airborne diseases due to the presence of multiple users, some of whom may be infected with such diseases.

After the outbreak of COVID-19, most of the research works focused on reducing the spread of the virus. Several methods are proposed for COVID-19 risk assessment, mainly to find the risk in an infected area. One such study, “Assessment of covid-19 risk and prevention effectiveness among spectators of mass gathering events,” by Murukami et al, 2022, discloses a model to reduce the risk of COVID-19 in Tokyo Olympics, in which infection risk and the number of infected persons was calculated using Monte Carlo simulation and preventive measures were suggested to reduce the infection risk in mass gatherings, such as in sports. Their environmental exposure model assessed infection risk and evaluated the effectiveness of various preventive measures. The inputs used for the study were merely the emissions from the person by talking, coughing, sneezing, and hand contact, without taking into consideration the health condition of the infected person to calculate the infection risk.

Another study “Research on risk assessment model of epidemic diseases in a certain region based on Markov chain and AHP”, by Yang et al., 2021 proposed a model for epidemic risk assessment by combining the Markov chain and Analytic Hierarchy Process (AHP). This model predicts a region's epidemic risk, which helps to get control over the epidemic in that region. However, the proposed model suits cities with a stable population, whereas there is a movement of people in a real scenario. Moreover, the proposed model is exclusively for COVID scenario and has not been considered for other such infectious diseases.

Another study “Infectious diseases surveillance in Pakistan: challenges, efforts, and recommendations,” by Bilal et al. 2022, discussed the diseases, HIV/AIDS, hepatitis B, and C, and tuberculosis which are the leading causes of morbidity and mortality in Pakistan. The study dealt with the reason for the spread of disease and measures to improve health, like educational awareness and financial support. It did not study the methods to assess the risk of spread of these diseases.

The study “A dynamic risk score to identify increased risk for heart failure decompensation,” by Sarkar et al, 2012 assesses the risk of patients having cardiac problems using the clustering technique based on vital signs. The method uses Bayesian Belief Network (BBN) for the risk assessment. BBN combines all features, generates the risk score, and determines whether the patient needs hospitalization. All patients are implanted with a device to collect their vital signs required for calculating the risk.

However, the implantation causes difficulty for the patients. Furthermore, the device implantation in each patient increases the overall cost of the risk assessment method.

Another study “Explainable machine learning for early assessment of COVID-19 risk prediction in emergency departments, by E. Casiraghi et al., 2020 proposed a machine learning-based system for predicting the risk of COVID-19 patients in the emergency department of a hospital. Clinical, laboratory, and radiological data of patients are used by Random Forest (RF) method to predict the risk. Nevertheless, preserving the privacy of patients is a challenge in this method. Although we are using the patients' medical conditions as secondary risk factors in our work, we are not using the details of the diseases as such. Therefore, privacy will not be an issue.

In a study “Development and validation of a web-based severe covid-19 risk prediction model,” by Woo et al., 2021, a COVID-19 risk prediction model was developed for hospitalized patients. Medical data provided in EHR (Electronic Health Record) was the only parameter used for prediction, and the sample size was small, thus, reducing the prediction accuracy.

In a similar study “Towards applying internet of things and machine learning for the risk prediction of covid-19 in pandemic situation using naive bayes classifier for improving accuracy,” by Deepa et al., 2022, a method was proposed for predicting the risk of COVID-19 of a person, in which data from IoT devices, Random Forest, and Naive Bayes classifiers collectively perform prediction, without considering the person's medical condition.

The study “An autoregressive graph convolutional long short-term memory hybrid neural network for accurate prediction of covid-19 cases”, by Ntemi et al., 2022, proposed a hybrid AR-GCN-LSTM method called the AGL method for predicting the number of COVID-19 cases on the next day for each state in the USA. The data used was the cumulative counts of COVID-19 cases released by the New York Times.

The study “Risk assessment for covid-19 pandemic in taiwan,” by Jian et al., 2021 assessed the risk of Taiwan for early detection of COVID-19, thus avoiding lockdown. The Event-Based Surveillance (EBS) of the study collected data from international organizations, government official websites, scientific journals, news, social media, and the internet bulletin board for risk assessment, leading to only a qualitative assessment.

A landslide early warning system (LEWS) and forecasting landslides using machine learning algorithms was designed in a study “Enhancing the reliability of landslide early warning systems by machine learning,” by Thirugnanam et al., 2020. In which the system can forecast a landslide 24 hours before its occurrence.

Based on the previous studies, most researchers proposed methods for risk assessment of COVID-19 using limited assessment parameters, which reduce risk assessment accuracy. Moreover, while some studies evaluated the risk in a stable population, which is impossible in a realistic environment, still other studies dealt with qualitative risk assessment and risk evaluations based on online questionnaires, which reduce the evaluated risk exactitude. Many similar studies have predicted risk using cumulative data, and in most cases, the lead time of prediction is shorter.

Methods and systems for assessing risk of contagious airborne diseases have been studied in the past, under different conditions, which have their own limitations making them less effective economically, qualitatively and quantitatively. Most of the studies have been done exclusively for COVID-19 scenario, without being replicated for other contagious airborne diseases. Moreover, some of the studies utilize methods which are privacy-invading, non-cost effective and limited to either only primary or secondary cumulative data with small sample size, thus, making them less accurate.

### BRIEF SUMMARY OF THE INVENTION

The present invention relates to a dynamic, cloud-based, IoT-enabled risk assessment and prediction architecture using the real-time primary data and secondary data of multiple users in an intelligent building (IB), for assessing and predicting the risk of contagious airborne diseases in the said intelligent building (IB).

The present invention aims to provide a two-phase risk assessment architecture, Risk Assessment in Intelligent Building architecture (RAIB), based on several factors affecting the risk of spreading the airborne contagious disease in an intelligent building (IB). The intelligent building is divided into sub-regions, each sub-region having multiple users. The first phase RAIB-I assesses the risk based on the primary risk factors computed from the real-time data of multiple users in sub-regions of the intelligent building (IB), whereas the second phase RAIB-II extrapolates the data of RAIB-I with the secondary risk factors to assess the risk in the said intelligent building (IB). Then the intelligent building sub-regions are clustered on the basis of K-means clustering method and classified into high, medium and low risk regions, based on the evaluated absolute risk from RAIB-I and RAIB-II. Thereafter, alert and warning messages are disseminated based on the sub-region's low, medium, and high-risk classification.

Further, the present invention provides a three-layered risk assessment architecture based on RAIB, comprising of an edge layer, a fog layer and a cloud layer for collecting and evaluating the primary and secondary risk factors and assessing the risk of spread of contagious airborne diseases.

The present invention further provides a prediction model, based on Seasonal Autoregressive Integrated Moving Average (SARIMA) model, to predict the risk of spread of contagious airborne diseases in an intelligent building (IB). Risk prediction is beneficial in reducing future risk by taking appropriate actions for mitigating the risk. The risk assessment and prediction system of the present invention utilizes time series data taken at regular intervals, along with the historical data of the users in intelligent building, to predict the future risk in the said intelligent building (IB). The simulation study is performed on a selected hospital pharmacy area efficiently classified pharmacy sub-regions based on risk assessment and predicted the risk.

Accordingly, the system of the present invention provides for a dynamic, economic and non-invasive model for identifying a user, assessing his existing risk factor namely the primary risk factor, accessing secondary risk factor for repeat user(s) for a given sub-region. Thereafter, the system computes total number of users in a sub-region having similar infections, and accesses historical data for a pre-defined period of time and predicting risk of contagious airborne infections based on both the primary risk factors, secondary risk factors and historical data. Where there is no historical data of said users such as new users the primary risk factors are computed for the said prediction.

Primary risk factors are computed from the data collected in real time by way of Internet of Things (IoT) in the intelligent building and secondary data refers to historical data of repeat users stored in the system database. This is applicable to multiple users in the intelligent building (IB).

BRIEF DESCRIPTION OF THE SEVERAL VIEWS OF THE DRAWINGS

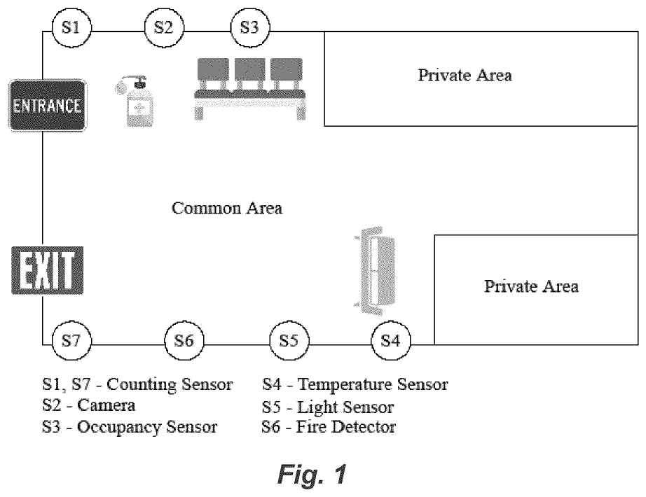

FIG. 1. depicts a selected area of the intelligent building with designated sub-regions based on user access such as common access or restricted access as in private area.

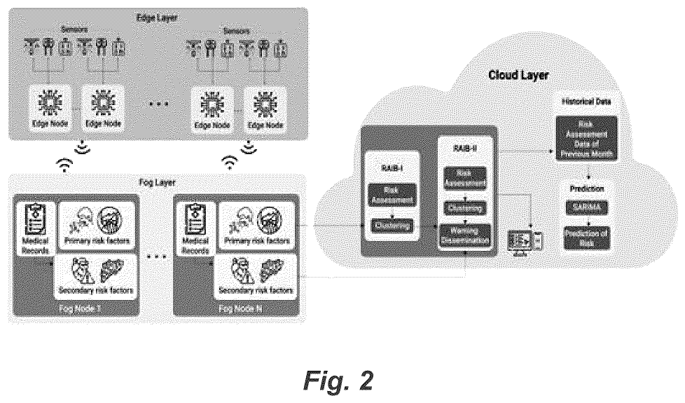

FIG. 2. depicts the two-phase, IoT based risk assessment and prediction RAIB architecture deployed in the three-layer IoT model, consisting of edge layer, fog layer, and cloud layer,

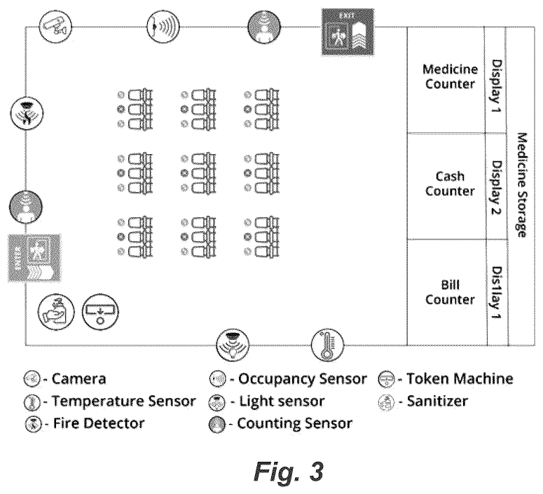

FIG. 3. depicts layout of the intelligent hospital pharmacy

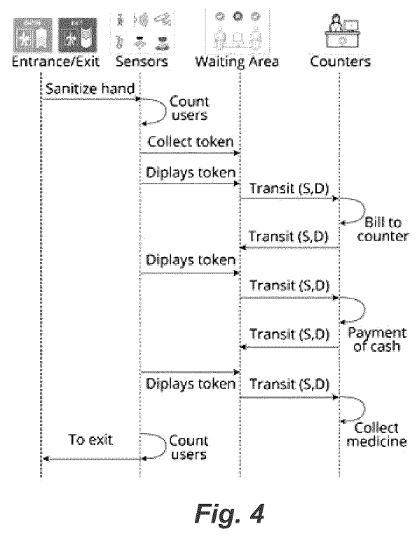

FIG. 4. illustrates activities of users of the pharmacy from the entrance to the exit

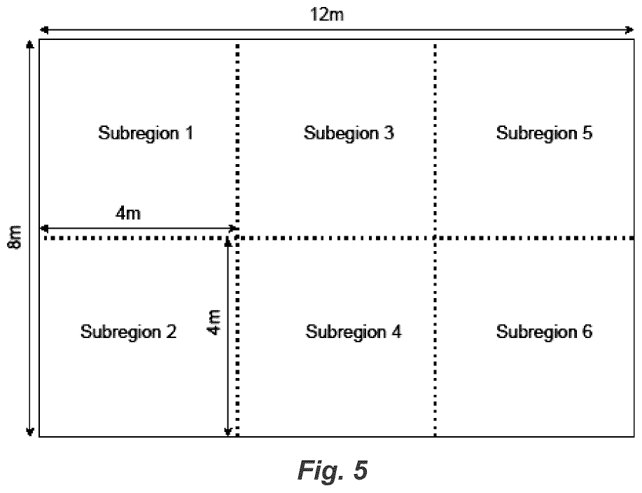

FIG. 5. depicts area of a pharmacy showing sub-regions

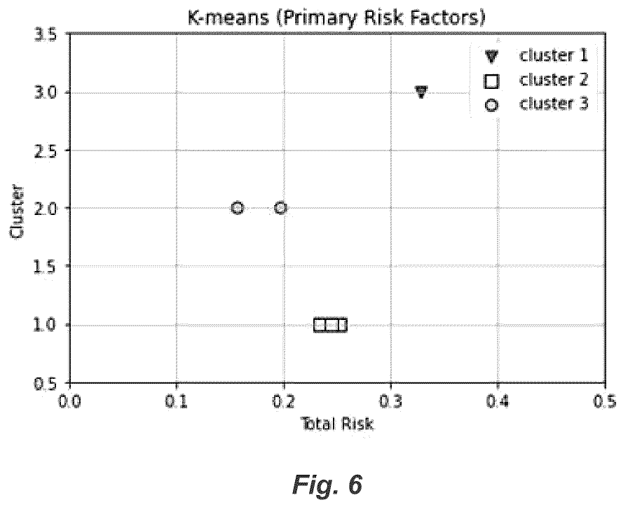

FIG. 6. depicts clustering of subregions into three clusters, cluster 1, cluster 2, and cluster 3

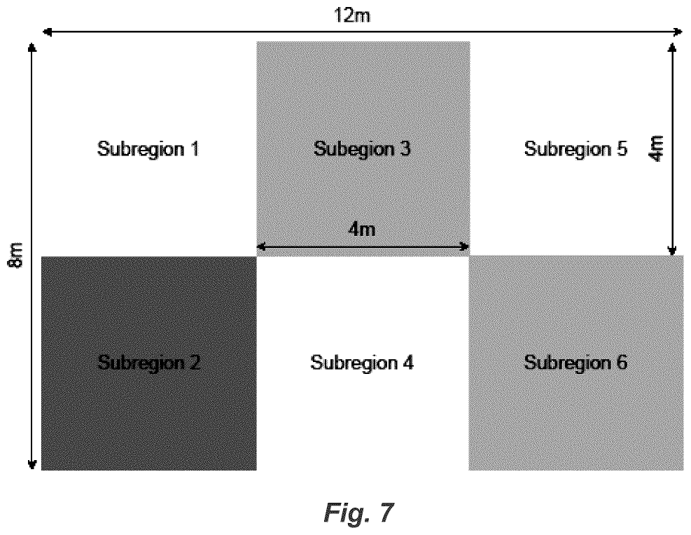

FIG. 7. depicts indication of RAIB-I showing the risk of subregions

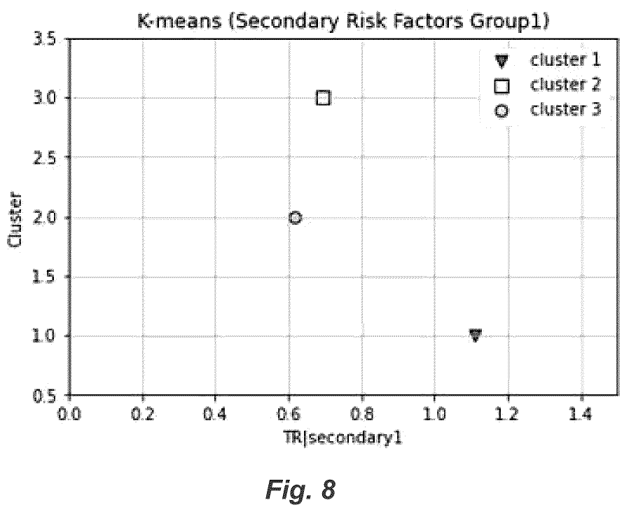

FIG. 8. depicts Result of RAIB-II Group1

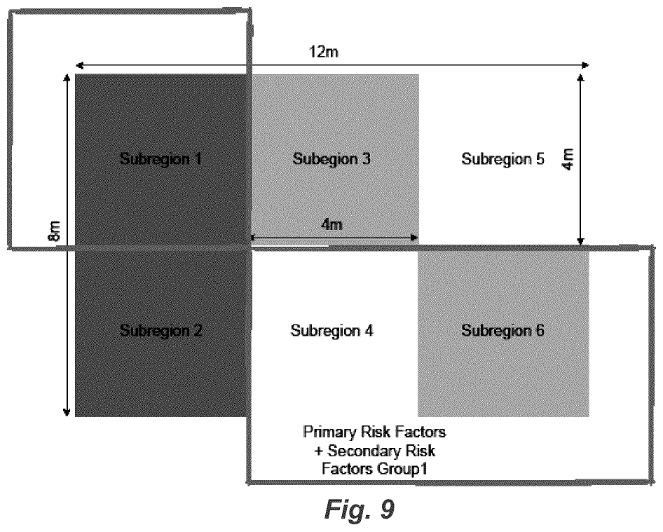

FIG. 9. depicts Indication of RAIB-II Group1

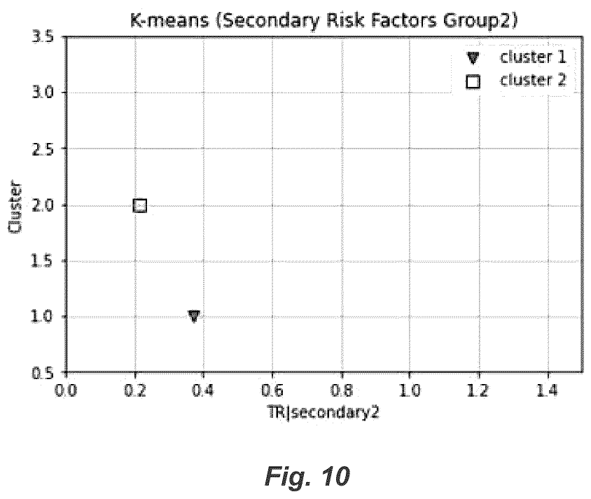

FIG. 10. depicts Result of RAHP-II Group2

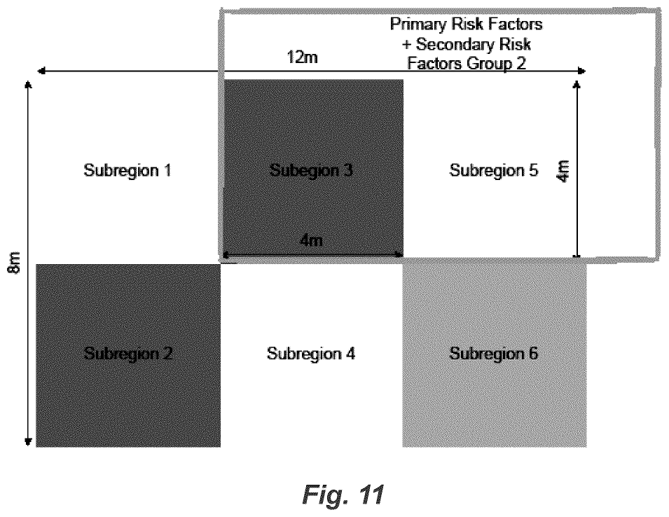

FIG. 11. depicts Indication of RAIB-II Group2

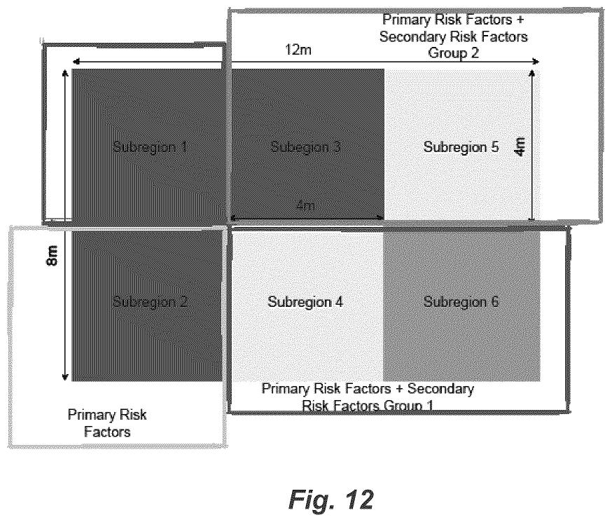

FIG. 12. depicts Indication of RAIB

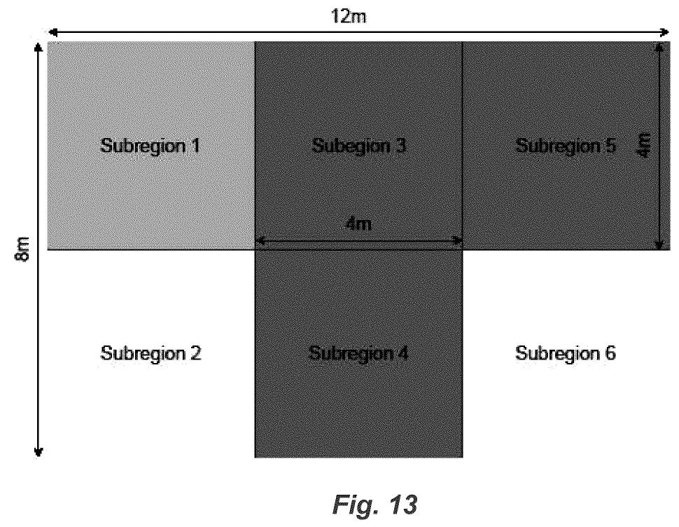

FIG. 13. depicts Indication of Prediction

### DETAILED DESCRIPTION OF THE INVENTION

The present invention in one embodiment discloses a cloud-based, IoT-enabled dynamic risk assessment and prediction system for assessment and prediction of contagious air-borne diseases in an intelligent building. The intelligent building is designated into sub-regions on the basis of user access such as, at least one common area and at least one private area.

An important object of the present invention is to provide a two-phase, IoT enabled, dynamic Risk Assessment and Prediction Architecture (RAIB), which assesses and predicts the risk of airborne contagious diseases in an intelligent building. Yet another object of the invention is to provide a two-phase, IoT enabled, Dynamic Risk Assessment and Prediction Architecture (RAIB) comprising of a risk assessment module I (RAIB-I) for assessing the risk based on primary risk factors obtained in real-time and a risk assessment module II (RAIB-II) for assessing the risk based on secondary risk factors, for each designated sub-region of the intelligent building.

Yet another object of the invention is to provide a two-phase RAIB deployed in a three-layered, IoT model comprising of edge layer, fog layer and cloud layer for collecting and analyzing the primary and secondary data for each sub-region, thus enabling the complete dynamic risk assessment and prediction of contagious airborne diseases in an intelligent building (IB).

Yet another object of the invention is to provide a prediction model, based on SARIMA (Seasonal Autoregressive Integrated Moving Average) model for predicting the future risk of intelligent building (IB), based on historical risk-assessment data.

Yet another object of the invention is to classify the sub-regions by K-means clustering method into high, medium and low risk subregions based on assessment of primary and secondary data of each sub-region.

Yet another object of the invention is to disseminate warnings to the high-risk sub-regions, based on the assessment of risk of contagious airborne diseases in the intelligent building (IB).

The present invention relates to a dynamic, cloud based, three-layered, IoT-enabled dynamic risk assessment and prediction system(S) for assessing as well as predicting the risk of spread of contagious airborne disease in an intelligent building (IB) on the basis of primary risk factors (fp1, fp2, fp3 . . . fpn) and secondary risk factors (fs1, fs2 . . . fsn).

Smart technologies as part of intelligent buildings (IBs) have evolved towards a strong integration of human, collective, and artificial intelligence. An Intelligent building utilize the integrated IoT-connected devices, cloud computing, augmented reality, and other systems to automate day-to-day processes. The advanced networks of intelligent building (IBs) use multiple sensors to detect the location and features of multiple users in different sub-regions of the building. Sensors may comprise of temperature sensors, light sensors, motion sensors, occupancy sensors, counting sensors, fire detectors, and cameras.

The term “building” used in the present invention refers to “Intelligent Building”, unless specified otherwise. The intelligent building (IB) has designated sub-regions (z1, z2, z3, . . . zn) which is based on the number and type of users who access such area. For instance, the reception or the waiting area would have all kinds of users and would therefore be a common area while the examination room or consultation room would have restricted access such as access by a patient to a doctor, said sub-region being designated as private area.

The present era witnessed the pandemic COVID-19, an airborne contagious disease that spreads quickly in crowded places. With the gradual rise of airborne infections around the world, there is a growing need to save the population from such infections. With the rapid industrialization and technological advancements, buildings are occupying the spaces which used to be open areas. Buildings include offices, housing societies, schools, hospitals, pharmacies etc. People visiting these buildings are more exposed to contagious airborne infections, due to their proximity with other people occupying the same space. In a building, presence of any person infected with a contagious airborne disease, increases the chances of other people, occupying the same space, from getting infected with such disease. More the number of people in an area of a building, greater the chance of rapid spread of infections. In a building, common areas are more prone to airborne contagious disease spread since almost everyone in the building or visiting the building may pass through the common area. Therefore, there is a relevant need to assess the risk of spread of airborne diseases and thus, issue warning in places where there is a possibility of exposure of people to such diseases.

The present invention discloses a dynamic, economic and non-invasive model for assessing and predicting risk of contagious airborne infections from both the primary risk factors (fp1, fp2, fp3 . . . fpn) and secondary risk factors (fs1, fs2, . . . fsn). Primary risk factors (fp1, fp2, fp3 . . . fpn) are data collected in real time by way of IoT in the intelligent building (IB) and secondary data refers to historical data (HD) of repeat users stored in the system database. This is applicable to multiple users (q1, q2, q3 . . . qn) in an intelligent building (IB). Where there is no historical data (HD) of said users such as new users the primary risk factors (fp1, fp2, fp3 . . . fpn) are computed for the said prediction.

Accordingly, the present invention provides a dynamic, two-phase, IoT-based system (S) and method for risk assessment and prediction of any airborne contagious disease in an intelligent building (IB). The features of the system (S) disclosed in the present invention include an IoT-based airborne contagious disease risk assessment architecture, Risk Assessment in Intelligent Building (RAIB), comprising of the first phase, RAIB-I, which assesses the risk based on the primary risk factors (fp1, fp2, fp3 . . . fpn) computed from the data of multiple users (q1, q2, q3 . . . qn) collected in real time, and the second phase, RAIB-II which extrapolates the data of RAIB-I with the secondary risk factors (q1, q2, q3 . . . qn) computed from the health data of repeat users stored in the database, a prediction-based module based on SARIMA (Seasonal Autoregressive Integrated Moving Average) model, which uses the historical risk assessment data (HD) to predict the future risk in the said intelligent building (IB), and numerical simulation analysis performed on a selected area of the intelligent building (IB) that reveals an efficient classification of sub-regions (z1,z2,z3 . . . zn) based on risk assessment and prediction.

The present invention provides for a dynamic, economic and non-invasive model for identifying user(s), assessing their existing risk factor namely the primary risk factor (fp1, fp2, fp3 . . . fpn), thereafter accessing secondary risk factors (fs1, fs2, . . . fsn) for repeat user(s) for a given sub-region (z1,z2,z3 . . . zn). Thereafter, the system(S) computes total number of users in a sub-region (z1,z2,z3 . . . zn) having similar infections, and accesses historical data (HD) for a pre-defined period of time and predicting risk of contagious airborne infections based on the primary risk factors (fp1, fp2, fp3, . . . fpn), the secondary risk factors (fs1, fs2 . . . fsn) and historical data (HD).

Primary risk factors (fp1, fp2, fp3 . . . fpn) are data collected in real time by way of IoT in the intelligent building (IB) and secondary data refers to historical data (HD) of repeat users stored in the system database. This is applicable to multiple users (q1, q2, q3 . . . qn) in the intelligent building (IB).

The layout of the selected area of the intelligent building (IB) is shown in FIG. 1.

The intelligent building is equipped with different sensors (sn1, sn2, sn3 . . . snn) to monitor and control the conditions inside the building. Temperature, light, and occupancy sensors help maintain the building's comfort level, whereas the counting sensors at the entrance and exit count the persons entering and leaving the area. Fire detectors detect the presence of fire, and cameras help to monitor the person entering the area. A person needs to sanitize with a sanitizer to prevent the spread of airborne contagious diseases. Then the person can go to the place wherever he wants other than any private area. However, the person can enter the private area with prior permission. The person can be seated in the allowed area until permission is granted. These sensors also detect the movement of people and collect data on the physical state of the people.

### RAIB: Risk Assessment in Intelligent Building

### (A) Risk Assessment Of Contagious Airborne Diseases:

The present invention discloses a two-phase risk assessment and prediction system (S), Risk Assessment in Intelligent Building (RAIB) based on the primary and secondary risk factors affecting the risk of contagious diseases spread. The RAIB architecture comprises of first phase RAIB-I which assesses the risk based on the primary risk factors (fp1, fp2, fp3, . . . fpn), and second phase RAIB-II which infers the risk assessment based on the secondary risk factors (fs1, fs2, . . . fsn). The output of the RAIB-I phase is used as input to the RAIB-II phase of the risk assessment. For the risk assessment of contagious diseases spread, the factors affecting contagious diseases spread have been categorized into primary and secondary risk factors. The primary risk factors (fp1, fp2, fp3, . . . fpn) considered are the number of users not correctly wearing the mask, the number of users coughing, sneezing, and talking, number of users making hand contact, number of persons in 1 m², temperature, air quality, touching face and presence of children. These risk factors are selected based on how the virus spreads from one person to another. The secondary risk factors (fs1, fs2 . . . fsn) considered are the medical history of users and if the users are vaccinated or not. Since the users with any other disease are more prone to contagious diseases than other individuals, the users' medical history has been selected as one of the secondary factors (fs1, fs2 . . . fsn) for higher risk assessment accuracy.

The RAIB architecture of the present invention uses the three-layer IoT model, comprising of edge (EL), fog (FL), and cloud layer (CL), integrated with each other, as shown in FIG. 2. The edge layer consists of sensors (sn1, sn2, sn3 . . . sn) integrated with

IoT edge devices, whereas the fog layer consists of fog nodes (fn1, fn2, . . . fnn) capable of processing and storing the data received from the edge layer. Data from the edge layer (EL) and fog layer (FL) are used in the cloud layer (CL) to perform the two-phase risk assessment, RAIB-I and RAIB-II, and risk prediction from the stored historical data

(HD). For ease of data collection and evaluation, the intelligent building has been divided into a number of sub-regions (z1, z2, z3, . . . zn).

The edge layer (EL) consists of the IoT-based Covi-RAIB architecture comprising of sensors integrated edge nodes (En1, En2, En3, . . . Enn). FIG. 2 depicts edge nodes corresponding to a given sensor. The RAIB architecture uses multiple homogenous and heterogenous sensors (sn1, sn2 . . . snn), already installed in intelligent buildings (IBs) to monitor several parameters of multiple users (q1, q2, q3, . . . qn) in the intelligent building (IB). Data is gathered from temperature sensors, light sensors, occupancy sensors, counting sensors, fire detectors, and cameras and process the data to obtain relevant information for risk assessment. In one such embodiment of the present invention, data has been collected from several cameras to obtain information on events such as coughing, sneezing, talking, wearing a mask, hand contact, and so on of a single user to be used by the IoT based RAIB architecture. Upon receiving the data from all such sensors and cameras, the edge nodes (En1, En2, En3, . . . . Enn) process the data and makes decisions favorable to each event. Therefore, the edge layer (EL) is scalable for multiple heterogeneous and homogenous sensors (sn1, sn2, sn3, . . . Snn), it aggregates the data from the sensors and transmits relevant information to the fog layer (FL).

A homogeneous sensor network consists of identical nodes, while a heterogeneous sensor network consists of two or more types of nodes (organized into hierarchical clusters).

The fog layer consists of fog nodes (Fn1, Fn2, Fn3, . . . Fnn) for receiving information regarding risk factors from the edge layer (EL) and health conditions stored in the database. After this, the fog nodes (Fn1, Fn2, Fn3, . . . Fnn) classify the risk factors into primary and secondary risk factors, as shown in Tables I, II. A fog node (FN) is connected with many edge nodes (En1, En2, En3, . . . Enn) in the edge layer (EL). Therefore, fog nodes (Fn1, Fn2, Fn3, . . . . Fnn) require more computational power than edge nodes (En1, En2, En3, . . . . Enn). The classified risk factors from fog nodes (Fn1, Fn2, Fn3, . . . Fnn) are transmitted to the cloud layer (CL) for risk assessment and prediction.

TABLE I
Primary Risk Factors

| SI. No. | Primary Risk Factors |
|---|---|
| 1 | Coughing without properly wearing the mask (CG\|NM) |
| 2 | Sneezing without properly wearing the mask (SN\|NM) |
| 3 | Talking without properly wearing the mask (TK\|NM) |
| 4 | Not wearing the mask properly (NM) |
| 5 | Making hand contact (HC) |
| 6 | Number of person in 1 m² (NS) |
| 7 | Temperature (TM) |
| 8 | Air quality (AQ) |
| 9 | Touching face (TF) |
| 10 | Presence of children (PC) |

TABLE II
Secondary Risk Factors

| SI. No. | Secondary Risk Factors |
|---|---|
| 1 | Cardiovascular disease (CV) |
| 2 | Nephropathy (NP) |
| 3 | Cancer (CA) |
| 4 | Cirrhosis (CH) |
| 5 | Asthma (AM) |
| 6 | Chronic obstructive pulmonary disease (CP) |
| 7 | Not vaccinated (NV) |
| 8 | Pregnancy (PN) |
| 9 | Chronic respiratory disease (CR) |
| 10 | Hematological malignancy (HM) |

The cloud layer (CL) receives data pertaining to the primary and secondary risk factors from fog nodes (Fn1, Fn2, Fn3, . . . Fnn) of the fog layer (FL). A two-phase risk assessment model, i.e., risk assessment and risk prediction model, Risk Assessment in Intelligent Building (RAIB) and a risk prediction-based model, such as SARIMA model are deployed in the cloud layer (CL). The RAIB model further comprises of RAIB-I which collects primary risk factors from sensors deployed in the edge layer (EL) and RAIB-II which gathers the secondary risk factors from the fog layer (EL) and extrapolates it with the historical data (HD) to provide risk assessment.

The sub-regions (z1, z2, z3, . . . zn) are clustered based on the computed values of risk from the risk factors collected from each sub-region (z1, z2, z3, . . . zn) of the intelligent building (IB). The major modules in the cloud layer (CL) are RAIB-I, RAIB-II for risk assessment and a risk prediction model, along with a database for storing sub-region-wise historical risk values and graphical user interfaces for users. In the RAIB-I risk assessment phase, primary risk factors (fp1, fp2, fp3, . . . fpn) are used to assess the risk for each subregion. Depending on the value of the primary risk factors, the risk may vary for different subregions (z1, z2, z3, . . . zn).

The intelligent building sub-regions (z1, z2, z3, . . . zn) are clustered and classified based on the absolute risk evaluated by RAIB architecture.

The risk of airborne contagious disease spread is assessed and clustered for each sub-region (z1, z2, z3, . . . zn) using the K-means clustering method, by using the data from

RAIB-I and RAIB-II. The sub-regions (z1, z2, z3, . . . zn) are classified as low, medium and high-risk sub-regions. Thereafter, alert and warning messages are disseminated based on the subregion's low, medium, and high-risk classification.

Even though the IoT-based RAIB model is proposed to assess the risk in a selected area, the same model can be extended to other intelligent rooms and buildings for airborne contagious disease risk assessment.

### RAIB-I

In risk assessment phase-I, RAIB-I, the subregions (z1, z2, z3, . . . zn) of an intelligent building (IB) are classified as low, medium and high-risk based on the primary risk factors (fp1, fp2, fp3, . . . fpn).

For easy risk assessment and dissemination of warnings, each region is divided into z equal subregions (z1, z2, z3, . . . zn). The first phase of risk assessment is used to assess the risk of a region based on the calculated risk of each subregion of that region.

Sensors (Sn1, Sn2 . . . , Snn) installed in a region collect data for the primary risk factors for that region. The primary risk factors (fp1, fp2, fp3, . . . fpn) selected for each subregion (z1, z2, z3, . . . zn) in RAIB-I are given in Table 1. These selected primary risk factors (fp1, fp2, fp3, . . . fpn) have a significant influence on the spread of the virus. The output of sensors is then used to assess the risk associated with the region. A rank (R) is assigned to each risk factor, which is based on the ability of the risk factor to cause the spread of the virus.

Each risk factor has a risk value (RV). The Risk value (RV) finds the weight of each risk factor using Direct Weight Elicitation Techniques, impact value (IV), and risk value (RV).

The risk value of a risk factor is calculated as the product of the probability of the risk factor, P (RF) and the impact value of the risk factor IV (RF) as shown in equation (1):

$$\\ \begin{matrix}RV = P ( RF ) * IV ( RF ) . & ( 1 )\end{matrix}$$

In the next step, the probability of each primary risk factor is calculated using the equation (2):

$$\\ \begin{matrix}Probability ( primary risk factor ) = Probability ( favorable outcome ) / Probability ( total outcome ) . & ( 2 )\end{matrix}$$

Subsequently, the impact value of each primary risk factor (fp1, fp2, fp3, . . . fpn) is calculated as the product of the probability and weight of the corresponding primary risk factor, which is shown in equation (3):

$$\\ \begin{matrix}Impact Value ( primary risk factor ) = Probability ( primary risk factor ) * Weight ( primary risk factor ) . & ( 3 )\end{matrix}$$

Now, the weight of the primary risk factor (fp1, fp2, fp3, . . . fpn) is calculated using Direct Weight Elicitation Techniques, as given in equation (4):

$$\\ \begin{matrix}{wt}_{i} = \frac{K - {r}_{i} + 1}{{\sum }_{j = 1}^{K} K - {r}_{j} + 1} & ( 4 )\end{matrix}$$

where, K depicts the total number of risk factors; ri depicts Rank of ith risk factor and rj depicts rank of the jth risk factor

The risk value for each primary risk factor (fp1, fp2, fp3, . . . fpn) is calculated as shown in equation (5):

$$\\ \begin{matrix}Risk Value ( primary risk factor ) = Probability ( primary risk factor ) * Impact Value ( primary risk factor ) . & ( 5 )\end{matrix}$$

Lastly, the total risk value of a subregion (z1, z2, z3, . . . , zn) is calculated, which is the sum of risk value due to each primary risk factor (fp1, fp2, fp3, . . . fpn) in that subregion as given by the equation (6):

$$\\ \begin{matrix}TRV ( primary ) = RV ( CG \vert NM ) + RV ( SN \vert NM ) + RV ( TK \vert NM ) + RV ( NM ) + RV ( HC ) & ( 6 )\end{matrix}$$

After finding the total risk value of all subregions (z1, z2, z3, . . . zn), the subregions (z1, z2, z3, . . . zn) are clustered using the K-means clustering method. Based on the total risk value, the subregions (z1, z2, z3, . . . zn) are classified as high, medium, and low-risk subregions.

### RAIB-II

Data evaluation and clustering of subregions (z1, z2, z3, . . . zn) in RAIB-I is followed by data assessment by RAIB-II. Before that, the subregions (z1, z2, z3, . . . zn) found with high risk in RAIB-I are omitted for RAIB-II because they are already at high risk, and the secondary risk factors are not going to decrease it. The low and medium-risk subregions are used to calculate the risk value due to secondary risk factors (fs1, fs2, fs3, . . . fsn).

In the second phase risk assessment, RAIB-II, the health database, which contains data on secondary risk factors (fs1, fs2 . . . fsn) is used to calculate the risk along with the risk assessment of RAIB-I. The secondary risk factors (fs1, fs2, fs3, . . . fsn) as considered for the present invention have been given in Table II. The subregions (z1, z2, z3, . . . zn) are grouped other than the high-risk subregions, based on the presence of secondary risk factors (fs1, fs2 . . . fsn). Since in some subregions (z1, z2, z3, . . . zn), the secondary factors (fs1, fs2 . . . fsn) are zero, therefore, the subregions (z1, z2, z3, . . . zn) are grouped with similar secondary risk factors (fs1, fs2, . . . fsn).

The same subregions (z1, z2, z3, . . . , zn) are grouped with the secondary risk factors (fs1, fs2 . . . fsn). For each group, the probability (P), impact value (IV), and risk value (RV) of secondary risk factors (fs1, fs2, fs3, . . . fsn) are calculated for the subregions included in the second phase, as shown in Table VIII. Direct Weight Elicitation Techniques, as used in RAIB-I is used to calculate the weight of secondary risk factors (fs1, fs2, fs3, . . . fsn). The total risk value for each subregion is calculated using the equation (7):

$$\\ \begin{matrix}TRV ( secondary ) = TRV ( primary ) + RV ( CV ) + RV ( NP ) + RV ( CA ) + RV ( CH ) + RV ( AM ) + RV ( CP ) + RV ( NV ) . & ( 7 )\end{matrix}$$

The subregions (z1, z2, z3, . . . zn) are then clustered and classified into high, medium, and low-risk regions using the K-means clustering method.

After the two-phase risk assessment, all the subregions (z1, z2, z3, . . . zn) are finally classified as high, medium, and low-risk subregions. Warnings can then be disseminated to the high-risk subregions based on the calculated risk assessment.

The algorithm used for risk assessment is given as Algorithm 1. The total area of the region is the input to the algorithm.

Algorithm 1 Risk Assessment:

Input: Total area of the region
Output: Classification of subregion into high, medium or low-risk region

| | |
|---|---|
| 1: | A = input (′total area of the region:′) |
| 2: | t = total number of risk factors |
| 3: | m = number of primary risk factors |
| 4: | n = number of secondary risk factors |
| 5: | g = number of secondary groups |
| 6: | Primary risk factors = fp1to fpm |
| 7: | Secondary risk factors = fs1 to fsn |
| 8: | SUBREGIONS(A) |
| 9: | Divide to z equal subregions |
| 10: | return z |
| 11: | OUTPUT OF SENSORS (fp1to fpm) |
| 12: | return fp1to fpm |
| 13: | HEALTH DATABASE (fs1 to fsn) |
| 14: | return fs1 to fsn |
| 15: | RANK (r_{f}p1 to r_{f}pm and r_{f}s1 to r_{f}sn) |
| 16: | return r_{f}p1 to r_{f}pm and r_{f}s1 to r_{f}sn |
| | (rfp=Rank of primary risk factors and rfs=Rank of secondary risk |
| | factors |
| 17: | OUTCOME (favorable outcome, total outcome) |
| 18: | P = N(fo) / N(o) |
| | (P=Probability; N(fo)=Number of favorable |
| | outcomes and N(o)=Total number of outcomes) |
| 19: | return P |
| 20: | RISK VALUE (risk factors, rank, P) |
| 21: | |
| | wₜ = number of risk factors − rank of ith risk factor |
| | +1 / Σⱼ₌₁^{numberofriskfactors}number of risk factors − rank |
| | of ith risk factor +1 |
| 22: | IV = P * wₜ |
| 23: | RV = P * IV |
| 24: | return RV |
| 25: | CLUSTERING (TRV) |
| 26: | Clusters the sub regions |
| 27: | Label the clusters as high, medium and low-risk |
| 28: | return Label |
| 29: | Call OUTPUT OF SENSORS (f p1 to f pm) |
| 30: | for h = 0; h < z; h++ do |
| 31: | TRV(i) = 0 |
| 32: | for i = 0; i < m; i++ do |
| 33: | Call OUTCOME (f p1 to f pm) |
| 34: | Call RISK VALUE (f p1 to f pm) |
| 35: | TRV(h) = TRV(i) + RV(i) |
| 36: | end for |
| 37: | end for |
| 38: | Call CLUSTERING (TRV(i)) |
| 39: | Call HEALTH DATABASE (f s1 to fsn) |
| 40: | Group the subregions having same secondary factors |
| 41: | for l = 0; l < g; l++ do |
| 42: | for p = 0; p < z; h++ do |
| 43: | for k = 0; k < n; k++ do |
| 44: | Call OUTCOME (fs1 to fsn) |
| 45: | Call RISK VALUE (f s1 to fsn) |
| 46: | TRV(k) = TRV(i) + RV(k) + TRV(k) |
| 47: | end for |
| 48: | end for |
| 49: | end for |
| 50: | Call CLUSTERING (TRV(p)) |
| 51: | Save to database |

### (B) Risk Prediction Model

Along with risk assessment, predicting future risk is very helpful in reducing the risk of each of the subregions (z1, z2, z3, . . . zn). In the present invention, SARIMA (Seasonal Autoregressive Integrated Moving Average) model is used to predict future risks in the selected area. Once the risk is predicted as high or medium in any subregion (z1, z2, z3, . . . zn), necessary actions can be taken to mitigate the spread of airborne contagious disease.

Therefore, in addition to the risk assessment, IoT-based RAIB architecture has a module for predicting future risks in the selected area. Risk assessment is performed every day using RAIB-I and RAIB-II for each sub-region (z1, z2, z3, . . . zn). Thereby obtained historical data (HD) on risk assessments for the intelligent building (IB) is used for predicting future risk of airborne contagious disease spread. Once the prediction module predicts the risk of airborne contagious disease spread, appropriate measures can be taken to reduce airborne contagious disease spread in the intelligent building (IB).

### Prediction of Future Risk

Historical data is taken for risk prediction and is available in the cloud layer (CL). The algorithm for prediction is shown as Algorithm 2.

Algorithm 2 Prediction of Future Risk.

Input: Historical risk assessment data of each subregion
Output: Predict the risk of each subregion

| | |
|---|---|
| 1: | z = number of subregions |
| 2: | PREDICTED TOTAL RISK VALUE (T RV ) |
| 3: | Estimate model parameters which gives lowest AIC |
| 4: | return Predicted TRV |
| 5: | CLUSTERING (Predicted TRV) |
| 6: | Clusters the sub regions |
| 7: | Label the clusters as high, medium and low-risk |
| 8: | return Risk |
| 9: | for h = 0; h < z; h++ do |
| 10: | Dickey-Fuller Test |
| 11: | if Data is non-stationary then |
| 12: | Differencing |
| 13: | Call PREDICTED TOTAL RISK VALUE(T RV ) |
| 14: | else |
| 15: | Call PREDICTED TOTAL RISK VALUE (T RV) |
| 16: | end if |
| 17: | end for |
| 18: | Call CLUSTERING (Predicted TRV) |
| 19: | Corresponding risk is the predicted risk |

For every sub-region (z1, z2, z3, . . . zn), historical risk assessment data is taken for one month. Firstly, the stationarity of the data is checked using the Dickey-Fuller test (DFT). If the data is non-stationary, it transforms it to stationary by differencing. Then, the Predicted total risk value (TRV) is evaluated, which first estimates the parameters of the SARIMA model. The model selects parameters with the lowest Akaike's Information Criterion (AIC) and fits the model with the data. After that, the model predicts the future total risk value for every subregion (z1, z2, z3, . . . zn). Then for every subregion, the Clustering (C) is evaluated, which clusters the predicted total risk value and classifies it as high, medium, and low-risk.

### Risk Assessment and Prediction in Intelligent Hospital Pharmacy

In a study conducted during the COVID-19 pandemic, the dynamic, two-phase, IoT-based risk assessment and prediction architecture, of the present invention, has been evaluated in an intelligent hospital pharmacy framework. The recent outbreak of COVID-19 pandemic caused widespread loss of life due to the highly contagious nature of the deadly COVID-19 virus, which spread quickly around cities and countries. Keeping in mind the high contagious airborne spread of COVID-19 in a crowded area, the study is based on an intelligent hospital pharmacy for COVID-19 for risk assessment and prediction.

COVID-19 is an airborne disease that spreads quickly in crowded places, which is why its risk assessment is more relevant in a crowded space. A hospital is such a spot where many people, including patients and their bystanders, visit, and a high degree of entry restrictions may not be practical. Furthermore, most hospital visitors are patients, which increases the risk of COVID-19 infection and spread. In a hospital, common areas, particularly pharmacies, are more prone to COVID-19 spread since almost everyone visiting the hospital may arrive at the pharmacy to collect medicines. Therefore, a hospital pharmacy scenario has been selected for COVID-19 risk assessment and prediction.

### Intelligent Hospital Pharmacy Framework

The layout of the intelligent hospital pharmacy is shown in FIG. 3. The intelligent pharmacy division has different sensors (Sn1, Sn2. . . . Snn) to monitor and control the conditions inside the pharmacy. Temperature, light, and occupancy sensors help maintain the pharmacy's comfort level, whereas the counting sensors at the entrance and exit count the persons entering and leaving the hospital pharmacy. Fire detectors detect the presence of fire, and cameras help to identify the proper wearing of the mask. Upon reaching the pharmacy, a person needs to sanitize with a sanitizer to prevent the spread of COVID-19. A person entering the pharmacy has to take a token number from the token machine since it is assumed that the service of the hospital pharmacy is token-based. Thereafter, the person is supposed to wait in the waiting area provided with chairs. However, the COVID-19 situation restricts the users from sitting in the middle chair to maintain physical distance. Three counters for bill distribution, cash collection, and medicine dispensing are in the hospital pharmacy and behind-the-counter stores medicine. Since the hospital pharmacy follows a token-based system, the display shows the token number at the respective counter, making the hospital pharmacy operations easier.

### Activity Sequence Diagram

The activities of users of the pharmacy from the entrance to the exit are illustrated in FIG. 4. At the entrance, the users sanitize their hands and enters the pharmacy. Then the user passes through the counting sensor, which counts the number of users joined in the pharmacy, and the camera helps to check whether the user is appropriately wearing the mask or not. Afterward, the user moves toward the token machine, pulls out the token number, and looks for a vacant chair in the waiting area. Then he waits for the token number to display at the respective counter. When the token number is displayed at the bill counter, the user collects it and pays the bill once the token number is displayed at cash counter. The user obtains the medicine when the token number is displayed at the medicine counter. Immediately after collecting the medicine, the user moves to the exit, and the counting sensor there counts down the user leaving the pharmacy.

### Numerical Analysis and Validation Based on Case Study

An IoT-based RAIB architecture has been used in the study, which includes a two-phase risk assessment model and SARIMA model-based prediction model. Additionally, the numerical analysis of the proposed risk assessment is performed and future risk is predicted.

### Simulation Scenario

The scenario taken for the risk assessment is the hospital pharmacy. For ease of calculation, we have assumed that the pharmacy is a rectangular region of area 96 m² (12×8 m), which is depicted in FIG. 5. The rectangular pharmacy region has been divided into six equal subregions of area 16 m² (4 m×4 m), which helps to focus on the high-risk subregions. Since COVID-19 has been chosen for the simulation purpose, the first five primary risk factors have been taken from Table I and the first seven secondary risk factors have been taken from Table II for risk assessment.

Python programming language has been used for the simulation of risk assessment and prediction of future risk. Risk assessment is done using the RAIB architecture and prediction is done using the SARIMA based prediction model. The total area z of the region taken into consideration is subdivided into equal subregions (z1, z2, z3, . . . zn. In the first phase of the risk assessment, RAIB-I, a rank is assigned to each primary risk factor, followed by calculation of probability (P), weight (W), impact value (IV), and risk value (RV) of the primary risk factors for each subregion. The risk values of all the subregions are summed up to receive the total risk value (TRV). These subregions are then clustered by the K-means clustering method, thus, classifying them into high, medium, and low-risk subregions.

In the second phase of risk assessment, RAIB-II, the medium and low-risk subregions are considered. A rank is assigned to each secondary risk factor (fs1,fs2, . . . fsn) and probability (P), weight (W), impact value (IV), and risk value (RV) of all secondary risk factors (fs1, fs2 . . . fsn) is calculated in each subregion.

Thereafter, the total risk value is calculated and subregions are clustered based on the total risk value of subregions (fs1, fs2 . . . fsn). Finally, the subregions are classified into high, medium, and low-risk subregions.

For predicting future risk, historical risk assessment data is used as the input. One-month risk assessment data for the simulation is taken to predict future risk. SARIMA model is used to predict every subregion's total risk value.

### Simulation Parameters

The intelligent building used for the study is equipped with all sensors (Sn1, Sn2. . . . Snn) for smooth functioning and comfort. Data is taken from these sensors for the purpose of risk assessment. Sensors (Sn1, Sn2, . . . . Snn) in the building give all the primary factors, and the hospital pharmacy database provides the patients' details, which are the secondary factors for calculating risk value. A rank is assigned to primary risk factors (fp1, fp2, fp3, . . . fpn), as given in Table III, based on the chance of spread of the virus.

Rank to secondary risk factors (fs1, fs2 . . . fsn) are given according to the seriousness of the medical condition and which are prone to the virus attack, as given in Table IV. Patients with cardiovascular disease, nephropathy, and cancer have the same risk and are given the same rank. Similarly, the secondary risk factors cirrhosis, asthma, and COPD are given the same rank.

TABLE III
Rank of Primary Risk Factors

| Risk Factor | Rank of Risk Factor |
|---|---|
| CG\|NM | 1 |
| SN\|NM | 2 |
| TK\|NM | 3 |
| NM | 4 |
| HC | 5 |

TABLE IV
Rank of Secondary Risk Factors

| Risk Factor | | Rank of Risk Factor |
|---|---|---|
| Medical History | Cardiovascular Disease | 1 |
| | Nephropathy | |
| | Cancer | |
| | Cirrhosis | 2 |
| | Asthma | |
| | COPD | |

| | |
|---|---|
| Not Vaccinated | 3 |

### RAIB System in Hospital Pharmacy

### RAIB-I

For analysis, the hospital pharmacy is divided into six equal subregions (z1, z2, z3, . . . z6). The total number of users considered are 500, out of which the number of users not wearing masks properly and making hand contact is between 251 and 500. The number of users coughing without wearing the mask, the number of users sneezing without wearing the mask, and the number of users talking without wearing the mask is taken between 1 and 250, as given in Table V.

Weight for each primary risk factor (fp1, fp2, fp3, . . . fpn) is calculated by Direct Weight Elicitation Techniques, shown in Table VI. Here, rank one is given to the primary risk factor (fp1, fp2, fp3, . . . fpn), coughing without properly wearing the mask because the droplets ejected during a cough are more than a sneeze. Using the values of primary risk factors from Table V, probability of each primary risk factor (fp1, fp2, fp3, . . . fpn) for all six subregions (z1, z2, z3, . . . z6) (Table IX), is calculated using the equations in Table VII. The impact value (IV) for each primary risk factor, which is the product of probability (P) and weight (W) of corresponding primary risk factors (fp1, fp2, fp3, . . . fpn), is calculated, as given in Table X.

TABLE V
Dataset of Primary Risk Factors

| Subregion | N(CG\|NM) | N(SN\|NM) | N(TK\|NM) | N(NM) | N(HC) |
|---|---|---|---|---|---|
| 1 | 173 | 48 | 118 | 444 | 481 |
| 2 | 193 | 68 | 196 | 290 | 338 |
| 3 | 104 | 10 | 212 | 425 | 339 |
| 4 | 22 | 243 | 37 | 332 | 416 |
| 5 | 88 | 71 | 217 | 276 | 328 |
| 6 | 89 | 141 | 59 | 323 | 260 |

TABLE VI
Weight of Primary Risk Factors

| wₜ(CG\|NM) | wₜ(SN\|NM) | wₜ(TK\|NM) | wₜ(NM) | wₜ(HC) |
|---|---|---|---|---|
| 0.333 | 0.266 | 0.2 | 0.133 | 0.066 |

TABLE VII
Probability, Impact Value and Risk Value of Primary Risk Factors

| Probability | Impact value | Risk Value |
|---|---|---|
| P(CG\|NM) = | IV(CG\|NM) = P(CG\|NM) * | RV(CG\|NM) = P(CG\|NM) * |
| (CG∩NM)/P(NM) (5) | wₜ (CG\|NM) (10) | IV(CG\|NM) (15) |
| P(SN\|NM) = | IV(SN\|NM) = P(SN\|NM) * | RV(SN\|NM) = P(SN\|NM) * |
| P(SN∩NM)/P(NM)(6) | wₜ (SN\|NM) (11) | IV(SN\|NM) (16) |
| P(TK\|NM)= | IV(TK\|NM) = P(TK\|NM) * | RV(TK\|NM) = P(TK\|NM) * |
| P(TK∩NM)/P(NM)(7) | wₜ (TK\|NM) (12) | IV(TK\|NM) (17) |
| P(NM) = NM/TU | IV(NM) = P(NM) * wₜ (NM) | RV(NM) = P(NM) * IV(NM) |
| (8) | (13) | (18) |
| P(HC) = HC/TU | IV(HC) = P(HC) * wₜ (HC) | RV(HC) = P(HC) * IV(HC) |
| (9) | (14) | (19) |

TABLE VIII
Probability, Impact Value and Risk
Value of Secondary Risk Factors

| Probability | Impact value | Risk Value |
|---|---|---|
| P(CV) = | IV(CV) = P(CV) * | RV(CV) = P(CV) * |
| CV/TP (20) | wₜ (CV) (27) | IV(CV) (34) |
| P(NP) = | IV(NP) = P(NP) * | RV(NP) = P(NP) * |
| NP/TP (21) | wₜ (NP) (28) | IV(NP) (35) |
| P(CA) = | IV(CA) = P(CA) * | RV(CA) = P(CA) * |
| CA/TP (22) | wₜ (CA) (29) | IV(CA) (36) |
| P(CH) = | IV(CH) = P(CH) * | RV(CH) = P(CH) * |
| CH/TP (23) | wₜ (CH) (30) | IV(CH) (37) |
| P(AM) = | IV(AM) = P(AM) * | RV(AM) = P(AM) * |
| AM/TP (24) | wₜ (AM) (31) | IV(AM)(38) |
| P(CP) = | IV(CP) = P(CP) * | RV(CP) = P(CP) * |
| CP/TP (25) | wₜ (CP) (32) | IV(CP) (39) |
| P(NV) = | IV(NV) = P(NV) * | RV(NV) = P(NV) * |
| NV/TP (26) | wₜ (NV) (33) | IV(NV) (40) |

TABLE IX
Probability of Primary Risk Factors

| Subregion | P(CG\|NM) | P(SN\|NM) | P(TK\|NM) | P(NM) | P(HC) |
|---|---|---|---|---|---|
| 1 | 0.389 | 0.108 | 0.265 | 0.888 | 0.962 |
| 2 | 0.665 | 0.234 | 0.675 | 0.580 | 0.676 |
| 3 | 0.244 | 0.023 | 0.498 | 0.850 | 0.678 |
| 4 | 0.066 | 0.731 | 0.111 | 0.664 | 0.832 |
| 5 | 0.318 | 0.257 | 0.786 | 0.552 | 0.656 |
| 6 | 0.275 | 0.436 | 0.182 | 0.646 | 0.520 |

TABLE X
Impact Value of Primary Risk Factors

| Subregion | IV(CG\|NM) | IV(SN\|NM) | IV(TK\|NM) | IV(NM) | IV(HC) |
|---|---|---|---|---|---|
| 1 | 0.129 | 0.028 | 0.053 | 0.118 | 0.064 |
| 2 | 0.221 | 0.062 | 0.135 | 0.077 | 0.045 |
| 3 | 0.081 | 0.006 | 0.099 | 0.113 | 0.045 |
| 4 | 0.022 | 0.195 | 0.022 | 0.088 | 0.055 |
| 5 | 0.106 | 0.068 | 0.157 | 0.073 | 0.043 |
| 6 | 0.091 | 0.116 | 0.036 | 0.086 | 0.034 |

The risk value (RV) for each primary risk factor (fp1, fp2, fp3, . . . fpn) is calculated as shown in Table XI, which is the product of probability (P) and impact value (IV). The total risk value in each subregion (z1, z2, z3, . . . zn) is calculated by summing up the risk value (RV) due to each primary risk factor (fp1, fp2, fp3, . . . fpn). This is followed by the clustering of subregions (z1, z2, z3, . . . zn), based on the total risk value (TRV), by the k-means clustering method. The number of clusters chosen are three, since the subregions have to be classified into low, medium, and high-risk. The clustering method clusters the subregions (z1, z2, z3, . . . zn) into three clusters, cluster 1, cluster 2, and cluster 3 which is shown in FIG. 6. The total risk value (TRV) of subregion 2 is high and is in cluster 1, whereas the total risk value (TRV) of subregion 3 and subregion 6 are low and are in cluster 3. The total risk value (TRV) of subregion 1, subregion 4, and subregion 5 is medium and is in cluster 2, as shown in Table XII.

Indication of RAIB-I in FIG. 7 clearly shows the risk of subregions (z1, z2, z3, . . . zn). Subregion 2 is at high risk, therefore it is excluded for the second level of risk assessment. Subregion 1, subregion 3, subregion 4, subregion 5, and subregion 6 are included for RAIB-II.

TABLE XI
Risk Value of Primary Risk Factors

| Subregion | RV(CG\|NM) | RV(SN\|NM) | RV(TK\|NM) | RV(NM) | RV(HC) |
|---|---|---|---|---|---|
| 1 | 0.050 | 0.003 | 0.014 | 0.105 | 0.061 |
| 2 | 0.147 | 0.014 | 0.091 | 0.044 | 0.030 |
| 3 | 0.019 | 0.000 | 0.049 | 0.096 | 0.030 |
| 4 | 0.001 | 0.142 | 0.002 | 0.058 | 0.046 |
| 5 | 0.033 | 0.017 | 0.123 | 0.040 | 0.028 |
| 6 | 0.025 | 0.050 | 0.006 | 0.055 | 0.018 |

TABLE XII
Total Risk Value and Risk of Subregions
due to Primary Risk Factors

| Risk | Subregion | TRV | Cluster |
|---|---|---|---|
| High | 2 | 0.328 | Cluster 1 |
| Medium | 1 | 0.234 | Cluster 2 |
| Medium | 4 | 0.251 | |
| Medium | 5 | 0.244 | |
| Low | 3 | 0.196 | Cluster 3 |
| Low | 6 | 0.156 | |

RAIB-II

For calculating the risk value (RV) due to secondary risk factors (fs1, fs2 . . . fsn), total number of patients is taken as 100. The dataset of secondary risk factors (fs1, fs2 . . . fsn) is given in Table XIII. Weight for each secondary risk factor (fs1, fs2 . . . fsn) is calculated using Direct Weight Elicitation Techniques, and rank is given, as indicated in Table IV. Calculated weights are shown in Table XIV.

TABLE XIII
Dataset of Secondary Risk Factors

| Sub- | | | | | | | |
|---|---|---|---|---|---|---|---|
| region | N(CV) | N(NP) | N(CA) | N(CH) | N(AM) | N(CP) | N(NV) |
| 1 | 20 | 80 | 69 | 79 | 47 | 64 | 82 |
| 2 | 99 | 88 | 49 | 29 | 19 | 19 | 14 |
| 3 | 0 | 0 | 65 | 9 | 57 | 32 | 31 |
| 4 | 74 | 23 | 35 | 75 | 55 | 28 | 34 |
| 5 | 0 | 0 | 36 | 53 | 5 | 38 | 17 |
| 6 | 79 | 4 | 42 | 58 | 31 | 1 | 65 |

TABLE XIV
Weight of Secondary Risk Factors

| wₜ (CV) | wₜ (NP) | wₜ (CA) | wₜ (CH) | wₜ (AM) | wₜ (CP) | wₜ (NV) |
|---|---|---|---|---|---|---|
| 0.5 | 0.5 | 0.5 | 0.333 | 0.333 | 0.333 | 0.166 |

There are total seven secondary risk factors (fs1, fs2 . . . fsn), but all these seven risk factors (fs1, fs2 . . . fsn) may not be in all subregions (z1, z2, z3, . . . zn). Some of the secondary risk factors (fs1, fs2 . . . , fsn) are zero in some subregions (z1, z2, z3, . . . zn). Therefore, the subregions (z1, z2, z3, . . . zn) with similar secondary risk factors (fs1, fs2 . . . fsn) are grouped together, since the subregions (z1, z2, z3, . . . zn) with different secondary risk factors (fs1, fs2. fsn) cannot be compared to determine the risk of the subregions (z1, z2, z3, . . . zn).

The subregions (z1, z2, z3, . . . zn) are divided into two groups. Group 1 consisting of subregion 1, subregion 4, and subregion 6, has all the seven secondary risk factors (fs1, fs2 . . . fsn), as given in Table XV. Subregion 3 and subregion 5 are in Group 2, as given in Table XX. The secondary risk factors CV and NP are zero for subregion 3 and subregion 5, and all other secondary risk factors (fs1, fs2 . . . fsn) are present in these subregions. The probability (P), impact value (IV) and risk value (RV) of secondary risk factors (fs1, fs2 . . . fsn) are calculated using the equations given in Table VIII.

### RAIB-II Group1

Group1 consist of subregion 1, subregion 4 and subregion 6, as given in Table XV. Firstly, the probability based on the secondary risk factors (fs1, fs2 . . . fsn) are calculated, as shown in Table XVI for the total number of patients. Then the impact value (IV), which is the product of the probability (P) and weight of secondary risk factors (fs1, fs2 . . . , fsn), is calculated for subregion 1, subregion 4, and subregion 6, as shown in Table XVII.

TABLE XV
Dataset of Secondary Risk Factors (Group1)

| Sub- | | | | | | | |
|---|---|---|---|---|---|---|---|
| region | N(CV) | N(NP) | N(CA) | N(CH) | N(AM) | N(CP) | N(NV) |
| 1 | 20 | 80 | 69 | 79 | 47 | 64 | 82 |
| 4 | 74 | 23 | 35 | 75 | 55 | 28 | 34 |
| 6 | 79 | 4 | 42 | 58 | 31 | 1 | 65 |

TABLE XVI
Probability of Secondary Risk Factors (Group1)

| Subregion | P(CV) | P(NP) | P(CA) | P(CH) | P(AM) | P(CP) | P(NV) |
|---|---|---|---|---|---|---|---|
| 1 | 0.20 | 0.80 | 0.69 | 0.79 | 0.47 | 0.64 | 0.82 |
| 4 | 0.74 | 0.23 | 0.35 | 0.75 | 0.55 | 0.28 | 0.34 |
| 6 | 0.79 | 0.4 | 0.42 | 0.58 | 0.31 | 0.01 | 0.65 |

TABLE XVII
Impact Value of Secondary Risk Factors (Group1)

| Sub- | | | | | | | |
|---|---|---|---|---|---|---|---|
| region | IV(CV) | IV(NP) | IV(CA) | IV(CH) | IV(AM) | IV(CP) | IV(NV) |
| 1 | 0.100 | 0.400 | 0.345 | 0.263 | 0.156 | 0.213 | 0.136 |
| 4 | 0.370 | 0.115 | 0.175 | 0.250 | 0.183 | 0.093 | 0.056 |
| 6 | 0.395 | 0.020 | 0.210 | 0.193 | 0.103 | 0.003 | 0.108 |

Further, Risk value (RV) is calculated for the three subregions (z1, z2, z3, . . . zn), which is the product of probability (P) and impact value (IV) as shown in Table XVIII.

For each subregion, risk value due to secondary risk factors (fs1, fs2 . . . fsn) is added to get TR|secondary1 (Total Risk Value for secondary risk factors in Group 1). This TRV due to secondary risk factors in Group 1, is then added with the total risk value (TRV) due to primary risk factors (fp1, fp2, fp3, . . . fpn) of the same subregions (z1, z2, z3, . . . zn). The sum obtained gives the combined risk value due to primary and secondary risk factors in subregion 1, subregion 4, and subregion 6, as shown in Table XIX. K-means clustering method is then applied, which clusters the subregions (z1, z2, z3, . . . zn) into three clusters, as depicted in FIG. 8. Subregion 1 is in cluster 1, subregion 4 in cluster 2, and subregion 6 in cluster 3. Then the subregion 1 is classified as high risk, subregion 4 as medium risk, and subregion 6 as low risk, as shown in Table XIX. Indication of RAIB-II Group1 shows that subregion 1 is at high risk, subregion 4 is at medium risk, and subregion 6 is at low-risk, as depicted in FIG. 9.

TABLE XVIII
Risk Value of Secondary Risk Factors (Group1)

| Subregion | RV(CV) | RV(NP) | RV(CA) | RV(CH) | RV(AM) | RV(CP) | RV(NV) |
|---|---|---|---|---|---|---|---|
| 1 | 0.020 | 0.320 | 0.238 | 0.208 | 0.073 | 0.136 | 0.112 |
| 4 | 0.273 | 0.026 | 0.061 | 0.187 | 0.100 | 0.026 | 0.019 |
| 6 | 0.312 | 0.000 | 0.088 | 0.112 | 0.032 | 0.000 | 0.070 |

TABLE XIX
Total Risk Value and Risk of secondary risk factors (Group1)

| Risk | Subregion | TRV\|secondary1 | Cluster |
|---|---|---|---|
| High | 1 | 1.343 | Cluster 1 |
| Medium | 4 | 0.946 | Cluster 2 |
| Low | 6 | 0.772 | Cluster 3 |

When we consider the secondary factors of Group1, the risk of subregion 1 is changed from medium to high, and subregion 4 and subregion 6 remain as such. This is illustrated in FIG. 7 and FIG. 9.

### RAIB-II Group2

In Group 2, subregion 3 and subregion 5 are included, as given in Table XX. The secondary risk factors CV and NP are zero in subregion 3 and subregion 5. The probability (P) of each secondary risk factor is calculated, as shown in Table XXI. The impact value (IV) is calculated as the product of probability (P) and weight (W), shown in Table XXII.

TABLE XX
Dataset of Secondary Risk Factors (Group2)

| Sub- | | | | | | | |
|---|---|---|---|---|---|---|---|
| region | N(CV) | N(NP) | N(CA) | N(CH) | N(AM) | N(CP) | N(NV) |
| 3 | 0 | 0 | 65 | 9 | 57 | 32 | 31 |
| 5 | 0 | 0 | 36 | 53 | 5 | 38 | 17 |

TABLE XXI
Probability of Secondary Risk Factors (Group2)

| Sub- | | | | | | | |
|---|---|---|---|---|---|---|---|
| region | P(CV) | P(NP) | P(CA) | P(CH) | P(AM) | P(CP) | P(NV) |
| 3 | 0.0 | 0.0 | 0.65 | 0.09 | 0.57 | 0.32 | 0.31 |
| 5 | 0.0 | 0.0 | 0.36 | 0.53 | 0.05 | 0.38 | 0.17 |

TABLE XXII
Impact Value of Secondary Risk Factors (Group2)

| Subregion | IV(CV) | IV(NP) | IV(CA) | IV(CH) | IV(AM) | IV(CP) | IV(NV) |
|---|---|---|---|---|---|---|---|
| 3 | 0.0 | 0.0 | 0.325 | 0.030 | 0.190 | 0.106 | 0.051 |
| 5 | 0.0 | 0.0 | 0.180 | 0.176 | 0.016 | 0.126 | 0.028 |

Risk value given in Table XXIII is the product of probability (P) and impact value (IV). Then total risk value (TRV) for the two subregions (z1, z2, z3, . . . zn) is calculated by adding the risk value (RV) of each secondary risk factor (fs1, fs2 . . . fsn). This is added to the total risk value (TRV) due to primary risk factors (fp1, fp2, fp3, . . . fpn) in these two subregions. Thus, the total risk value (TRV) for the two subregions is assessed. Then K-means clustering clusters the subregions (z1, z2, z3, . . . zn), and classifies them into high and medium-risk subregions. In this group, there are two subregions, subregion 3 is in cluster 1, and subregion 5 is in cluster 2, as depicted in FIG. 10. The subregion 3 is classified as high risk and subregion 5 as medium risk, as shown in Table XXIV. Indication of RAIB-II Group 2 is depicted in FIG. 11.

TABLE XXIII
Risk Value of Secondary Risk Factors (Group2)

| Subregion | RV(CV) | RV(NP) | RV(CA) | RV(CH) | RV(AM) | RV(CP) | RV(NV) |
|---|---|---|---|---|---|---|---|
| 3 | 0.0 | 0.0 | 0.211 | 0.002 | 0.108 | 0.034 | 0.016 |
| 5 | 0.0 | 0.0 | 0.064 | 0.093 | 0.000 | 0.048 | 0.004 |

TABLE XXIV
Total Risk Value and Risk of Secondary Risk Factors (Group2)

| Risk | Subregion | TRV\|secondary2 | Cluster |
|---|---|---|---|
| High | 3 | 0.569 | Cluster 1 |
| Medium | 5 | 0.456 | Cluster 2 |

Risk assessment of secondary factors of Group 2 shows that the risk of subregion 3 is changed from low to high, and subregion 5 is the same as the risk of primary risk assessment. This is understood by comparing FIG. 7 and FIG. 11.

After the two-phase risk assessment, subregion 1, subregion 2, and subregion 3 are assessed as high risk, subregion 4 and subregion 5 as medium risk, and subregion 6 as low risk, as depicted in FIG. 12.

### Prediction of Risk

Besides risk assessment, prediction of future risk is made by using the SARIMA (Seasonal Autoregressive Integrated Moving Average) model. For evaluation purposes, one-month risk assessment data is used for prediction. 70% of the data has been used for training and 30% for testing.

The model (0, 1, 1)*(3, 1, 0, 7) is the best, with the lowest AIC of −108.566. (Mean Average Error) MAE and (Mean Squared Error) MSE of the SARIMA model are 0.018 and 0.0008, respectively, which is a good predictor. The data used for prediction is the previous month's data for subregion 1, as given in Table XXV. Similarly, for all subregions, previous month's data is considered. The SARIMA model predicts the total risk value for every subregion (z1, z2, z3, . . . zn). Thereafter, K-means clustering clusters the subregions (z1, z2, z3, . . . zn) and classifies them as high, medium, and low-risk subregions. Thus, the risk of all the subregions is predicted, which is depicted in FIG. 13.

TABLE XXV
Risk Assessment Data used for Prediction
and Predicted Total Risk Value

| | | | Predicted Total Risk Value |
|---|---|---|---|
| Subregion | Day | Total Risk Value | for the Next 30 Days |
| 1 | Day 1 | 0.57 | 0.46 |
| | Day 2 | 0.84 | 0.66 |
| | Day 3 | 0.52 | 0.75 |
| | Day 4 | 0.46 | 0.95 |
| | Day 5 | 0.67 | 0.58 |
| | Day 6 | 0.91 | 0.84 |
| | Day 7 | 0.96 | 0.52 |
| | Day 8 | 0.59 | 0.46 |
| | Day 9 | 0.85 | 0.67 |
| | Day 10 | 0.52 | 0.73 |
| | Day 11 | 0.47 | 0.96 |
| | Day 12 | 0.68 | 0.57 |
| | Day 13 | 0.73 | 0.82 |
| | Day 14 | 0.97 | 0.53 |
| | Day 15 | 0.58 | 0.47 |
| | Day 16 | 0.82 | 0.66 |
| | Day 17 | 0.53 | 0.74 |
| | Day 18 | 0.48 | 0.96 |
| | Day 19 | 0.67 | 0.57 |
| | Day 20 | 0.75 | 0.84 |
| | Day 21 | 0.97 | 0.58 |
| | Day 22 | 0.58 | 0.46 |
| | Day 23 | 0.85 | 0.66 |
| | Day 24 | 0.59 | 0.74 |
| | Day 25 | 0.47 | 0.95 |
| | Day 26 | 0.67 | 0.57 |
| | Day 27 | 0.75 | 0.83 |
| | Day 28 | 0.95 | 0.52 |
| | Day 29 | 0.57 | 0.46 |
| | Day 30 | 0.84 | 0.66 |

The present invention discloses an automated risk assessment and prediction architecture, RAIB, which assesses the risk of an intelligent building (IB), thereby reducing the risk by taking necessary mitigation measures. The method, as disclosed, divides the focus region into different subregions (z1, z2, z3, . . . zn) and finds the risk of each. The system (S) comprises two phases of risk assessment, RAIB-I and RAIB-II. In

RAIB-I, the risk factors used are the primary risk factors (fp1, fp2, fp3, . . . fpn). In RAIB-II, the results of RAIB-I are used along with the secondary risk factors (fs1, fs2 . . . fsn) to calculate the risk value (RV). The subregions (z1, z2, z3, . . . zn) are clustered using the K-means clustering method and classified as high, medium, and low-risk regions. Thereafter, warnings are disseminated to high-risk subregions. Using historical risk assessment data, the risk of all subregions (z1, z2, z3, . . . zn) can be predicted, which can be used to mitigate the risk.

The risk assessment and prediction architecture as disclosed in the present invention can be replicated for risk assessment of any airborne contagious disease by choosing the primary and secondary risk factors, based on the disease. The invention is not limited to the primary and secondary risk factors, as disclosed. More primary risk factors and secondary risk factors, depending on the disease to be assessed, can be added to make the risk assessment and risk prediction better.

1. A cloud-based, IoT-enabled, dynamic risk assessment and prediction system (S) in an intelligent building, comprising:
Risk Assessment in Intelligent Building architecture (RAIB), being deployed in said intelligent building (IB) in a three-layered model comprising of edge layer (EL), fog layer (FL) and cloud layer (CL) wherein:
said edge layer (EL) comprising of at least one edge node (EN), each such edge node being integrated with multiple smart homogeneous and heterogeneous sensors (Sn1, Sn2 . . . Snn) installed with IoT edge devices within the common area of said intelligent building (IB), for obtaining primary risk factors (fp1, fp2, fp3, . . . fpn),
a fog layer (FL), wherein said fog layer (FL) comprising of at least one fog node (FN), each such fog node (FN) connected with multiple edge nodes (En1, En2 . . . Enn) for receiving, processing and storing information on risk factors from the edge layer (EL) and secondary risk factors (fs1, fs2 . . . fsn) comprising of the health conditions stored as historical data (HD),
a cloud layer (CL), wherein said cloud performs two-phase risk assessment based on primary risk factors (fp1, fp2, fp3, . . . fpn) and secondary risk factors (fs1, fs2 . . . fsn) from edge layer (EL) and fog layer (FL) and risk prediction from the said historical data (HD),
characterized in that the two-phase risk assessment and prediction architecture, RAIB, comprises of:
RAIB-I which assesses the risk based on primary risk Factors (fp1, fp2, fp3, . . . fpn),
RAIB-II which assesses the risk based on the secondary risk factors (fs1, fs2 . . . fsn), and
Seasonal Autoregressive Integrated Moving Average (SARIMA)-based prediction model (PM), which uses the historical risk assessment data to predict the future risk of spread of contagious airborne diseases in the said intelligent building (IB).

2. The risk assessment and prediction system (S), as claimed in claim 1, wherein the said intelligent building is divided into sub-regions (z1, z2, z3, . . . zn), with each such sub-region having multiple users (q1, q2, q3, . . . qn), such that the risk assessment and risk prediction is performed for such multiple users (q1, q2, q3, . . . qn), based on the risk assessment of sub-regions (z1, z2, z3, . . . zn).

3. The risk assessment and prediction system (S), as claimed in claim 1, wherein the sensors (Sn1, Sn2 . . . Snn) deployed in the edge layer (EL) of sub-regions (z1, z2, z3, . . . zn) of said intelligent building (IB) are selected from homogeneous or heterogeneous sensors (Sn1, Sn2 . . . Snn).

4. The risk assessment and prediction system (S), as claimed in claim 1, wherein homogeneous and heterogeneous sensors (Sn1, Sn2 . . . Snn) extract physical features (f=f1, f2, f3, . . . fn) of multiple users (q1, q2, q3, . . . qn) for each sub-region (z1, z2, z3, . . . zn), for purposes of categorizing as primary risk factors (fp1 . . . fpn) and health condition of said multiple users (q1, q2, q3, . . . qn) for creating primary health record of users present in a given sub-region.

5. The risk assessment and prediction system (S), as claimed in claim 1, wherein the fog layer (FL) accesses the health records of repeat users in said sub-region from the online database, for categorization of secondary risk factors (fs1, fs2, . . . fsn) of said user.

6. The risk assessment and prediction system (S) as claimed in claim 1, wherein, the data of primary risk factors (fp1, fp2, . . . , fpn) and secondary risk factors (fs1, fs2, . . . , fsn) of each sub-region (z1, z2, z3, . . . zn) are used by RAIB-I and RAIB-II in the cloud layer (CL) to cluster the sub-regions (z1, z2, z3, . . . zn), based on the assessed dynamic risk values (RV) of airborne contagious disease spread.

7. The risk assessment and prediction system (S) as claimed in claim 1, wherein the clustering of sub-regions (z1, z2, z3, . . . zn) is performed by using K-means clustering method and based on the total risk value (TRV) of each sub-region (z1, z2, z3, . . . zn), the sub-regions (z1, z2, z3, . . . zn) are clustered as low, medium and high-risk sub-regions.

8. The risk assessment and prediction system (S) as claimed in claim 1, wherein dynamic risk assessment is performed every day using RAIB-I and RAIB-II for each sub-region (z1, z2, z3, . . . zn),.

9. The risk assessment and prediction system (S) as claimed in claim 1, wherein risk prediction is performed by prediction model based on SARIMA ((Seasonal Autoregressive Integrated Moving Average) model which uses corresponding historical risk assessment data from the historical database (HD) in the cloud layer (CL) for a pre-defined period to predict the future risk in the said intelligent building (IB).

10. The risk assessment and prediction system (S) as claimed in claim 1, wherein the risk assessment data for ‘N’ weeks (range) is taken for the simulation to predict future risk of the intelligent building.

11. A Method of risk assessment and prediction of airborne contagious disease in the intelligent building (IB) as claimed in claim 1, comprising the steps of:
a) sub-dividing the total area of the intelligent building (IB) into equal sub-regions (z1, z2, z3, . . . zn), with each such sub-region having multiple users (q1, q2, q3, . . . qn),
b) extracting physical features (f=f1, f2, f3, . . . fn) of multiple users (q1, q2, q3, . . . qn) for each sub-region, by multiple homogeneous and heterogeneous sensors (Sn1, Sn2 . . . Snn) deployed in the edge layer (EL),
c) assessing the primary risk factors (fp1, fp2, fp3 . . . fpn) of each sub-region in the fog layer (FL) based on the data obtained from the edge layer (EL),
d) obtaining the secondary risk factors (fs1, fs2 . . . fsn) in the fog layer (FL) comprising of health conditions stored as historical data (HD),
e) assessing the primary risk factors (fp1, fp2, fp3, . . . fpn of said sub-regions (z1, z2,z3, . . . zn) by RAIB-I in the cloud layer (CL) by:
i. calculating the risk value of each primary risk factor (fp1, fp2 . . . fpn) in a sub-region (z1, z2, z3, . . . zn), using the probability (P) and impact value (IV) of each primary risk factor (fp1 . . . fpn) in the said sub-region (z1, z2, z3, . . . zn),
ii. assigning a rank to each primary risk factor (fp1, fp2, fp3, . . . fpn).
iii. calculating the total risk value (TRV) of the said sub-region (z1, z2, z3, . . . zn) by adding the risk value (RV) of each primary risk factor (fp1, fp2, . . . fpn) in the said sub-region (z1, z2, z3, . . . zn),
iv. calculating the probability (P), weight (W), impact value (IV), and risk value (RV) of each primary risk factor, for each subregion,
v. calculating the total risk value (TRV) for all subregions (z1, z2, z3, . . . zn),
vi. clustering the sub-regions (z1, z2, z3, . . . zn), using K-means clustering method,
vii. classifying the sub-regions (z1, z2, z3, . . . zn) as high, medium and low-risk sub-region,
f) considering the medium and low-risk subregions of RAIB-I for the second phase of risk assessment, RAIB-II, assigning a rank to each secondary risk factor (fs1, fs2 . . . fsn), calculating the probability (P), weight (W), impact value (IV), and risk value (RV) of all secondary risk factors (fs1, fs2 . . . fsn) in each subregion (z1, z2, z3, . . . zn),
g) calculating the total risk value (TRV) of all subregion (z1, z2, z3, . . . zn) due to secondary risk factors (fs1, fs2 . . . fsn) by RAIB-II, from the estimated low and medium-risk subregions of RAIB-I, extrapolating the value of secondary risk factors (fs1, fs2 . . . fsn) to it and classifying the subregions (z1, z2, z3, . . . zn) into low, medium and high-risk subregions,
h) adding the total risk value (TRV) of each subregion (z1, z2, z3, . . . zn) from RAIB-I and RAIB-II,
i) classifying each subregion (z1, z2, z3, . . . zn) into high, medium and low-risk subregions,
j) disseminating warning to high-risk subregions,
k) prediction of risk of contagious airborne infections of the said intelligent building (IB), in the cloud layer (CL) comprising the steps of:
collecting historical risk assessment data for one month,
checking the stationarity of the data using the Dickey-Fuller test,
calculating the Predicted total risk value (PRV), by first estimating the parameters of the SARIMA model,
selecting parameters with the lowest (Akaike's Information Criterion) AIC and fitting the model with the data,
predicting the future total risk value for every subregion (z1, z2, z3, . . . zn),
clustering (C) of the predicted total risk value (TRV) and classifying it as high, medium, and low-risk.
